(19) United States
(12) Patent Application Publication
(10) Pub. No.: US 2022/0395635 A1
(43) Pub. Date: Dec. 15, 2022

(54) DRUG DELIVERY SYSTEM AND METHOD OF USE

(71) Applicant: AMGEN INC., Thousand Oaks, CA (US)

(72) Inventor: Shawn P. Malloy, Cambridge, MA (US)

(21) Appl. No.: 17/770,349

(22) PCT Filed: Oct. 29, 2020
(86) PCT No.: PCT/US20/57821

§ 371 Date: Apr. 20, 2022

Related U.S. Application Data
(60) Provisional application No. 62/932,391, filed on Nov. 7, 2019.

(51) Int. Cl. A61M 5/168; A61M 5/152

(52) U.S. Cl. CPC A61M 5/16881; A61M 5/152; A61M 5/16813; A61M 5/16886; A61M 5/16831; A61M 2205/502

(57) ABSTRACT

A drug delivery system includes a delivery container including a container body adapted to accommodate a drug therein, a supply line, a flow rate sensor, and a flow controller. The delivery container further includes an outlet port and is constructed from a resilient material. The container body is adapted to exert an urging force on the drug to expel the drug from the outlet port. The supply line is operably coupled to the outlet port to deliver the drug to a user. The flow sensor senses a flow rate of the drug within the supply line. The flow controller is configured to regulate a flow rate of the drug; the flow controller includes a supply line restrictor and a controller for adjusting the supply line restrictor between at least two settings.

### CROSS-REFERENCE TO RELATED APPLICATIONS

Priority is claimed to U.S. Provisional Patent Application No. 62/932,391, filed Nov. 7, 2019, and the entire contents thereof are hereby incorporated herein by reference.

### FIELD OF THE DISCLOSURE

The present disclosure generally relates to drug delivery systems and methods and, more particularly, to improved approaches for preparing and delivering dosing systems.

### BACKGROUND

Drugs are administered to treat a variety of conditions and diseases. Intravenous therapy is a drug dosing process that delivers drugs directly into a patient's vein using an infusion contained in a delivery container (e.g., a pliable bag) and tubing connected to a needle subsystem that fluidically communicates with the reservoir through the pump assembly collectively called an infusion set. Similarly, infusion therapy may encompass IV therapy and/or delivery to subcutaneous or other tissue. The term “IV” as used herein shall be used to refer to intravenous and/or infusion therapies. In IV therapies, drug dosings may be performed in a healthcare facility, or in some instances, at remote locations such as a patient's home. In certain applications, a drug delivery process may last for an extended period of time (e.g., for one hour or longer) or may include continuous or semi-continuous delivery of a drug over an extended period of time (e.g., for several hours, days, weeks, or longer). For many of these relatively long-term delivery requirements, a pump is often utilized to control and/or administer the drug to the patient. The pump may be coupled (physically, fluidly, and/or otherwise) to various components, such as a drug delivery container, supply lines, connection ports, and/or the patient.

Alternatively, a disposable IV pump in the form of an elasticized balloon or an IV bag set for gravity drip may be used in an at-home setting to provide patients the ability to administer their own dosages. These take-home systems typically lack programming, are offered in a range of volumes and flow rates, and get lighter throughout delivery without the need for expensive maintenance and/or service infrastructure. However, oftentimes drugs in these disposable systems need to stay within a specific flow rate window, but they cannot alert a patient if the device becomes blocked or otherwise occluded. Compared to reusable systems, disposable systems generally do not rely on large, bulky electronics for proper operation, rather, these devices typically use their inherent elasticity to create a drug delivery pressure that, combined with tubing resistance, results in a predetermined drug flow rate. Conversely, reusable systems oftentimes have large power supplies that enable continued use for multiple days, and typically include a user interface having multiple, complex menus.

Oftentimes, a healthcare professional must prepare the drug by urging the drug into the bag using their own strength to overcome the inherent resistance of the bag, which can be difficult with drugs of varying viscosities and/or when filling bags, particularly balloon-style bags having relatively high elasticity. Any number of factors may impact the accuracy of the drug flow rate, as these devices typically lack programmability features. Additionally, the bags may be incapable of generating the required pressure near the end of a dosage cycle, and as a result, the patient may not receive the entire intended dosage.

As described in more detail below, the present disclosure sets forth systems and methods for patient monitoring and interventional dosing techniques embodying advantageous alternatives to existing systems and methods, and that may address one or more of the challenges or needs mentioned herein, as well as provide other benefits and advantages.

### SUMMARY

In accordance with a first aspect, a drug delivery system includes a delivery container including a container body adapted to accommodate a drug therein, a supply line, a flow rate sensor, and a flow controller. The delivery container further includes an outlet port and is constructed from a resilient material. The container body is adapted to utilize gravitational force to expel the drug from the outlet port. The supply line is operably coupled to the outlet port to deliver the drug to a user. The flow sensor senses a flow rate of the drug within the supply line. The flow controller is configured to regulate a flow rate of the drug; the flow controller includes a supply line restrictor and a controller for adjusting the supply line restrictor between at least two settings.

In some examples, the controller is adapted to receive the input from the flow rate sensor and compare the input to a desired flow rate to calculate a difference value. When the difference value exceeds a threshold value, the controller may be adapted to adjust an operational parameter of the supply line restrictor.

In some examples, the flow restrictor includes a ramped surface and a movable clamp, wherein a portion of the supply line is positioned between the ramped surface and the movable clamp, and wherein the flow controller includes a controller arm coupled with the movable clamp and configured to move the movable clamp between at least a first position and a second position.

In some examples, the flow restrictor includes a rotary dial, wherein a portion of the supply line is positioned adjacent to the rotary dial, and wherein the flow controller includes an eccentric portion coupled with the rotary dial and configured to move the rotary dial between at least a first position and a second position.

In some examples, the flow controller further includes an alarm operably coupled to the controller. In these examples, the controller may activate the alarm upon the difference value exceeding an alarm value. The alarm value may at least be partially based on a risk profile of the drug contained in the delivery container.

In some examples, the delivery container may be in the form of a balloon. The container body is adapted to exert an urging force on the drug to expel the drug from the outlet port. The supply line is operably coupled to the outlet port to deliver the drug to a user. In some examples, the supply line is in the form of spiral tubing. The flow rate sensor may be operably coupled to at least one of the delivery container or the supply line.

### BRIEF DESCRIPTION OF THE DRAWINGS

The above needs are at least partially met through provision of the continuous dosing system and approaches described in the following detailed description, particularly when studied in conjunction with the drawings, wherein:

FIG. 1 illustrates an example take-home, disposable drug delivery system in accordance with various embodiments;

FIG. 2 illustrates an example flow rate monitor of the example take-home, disposable drug delivery system of FIG. 1 in accordance with various embodiments;

FIG. 3 illustrates an exemplary flow controller for use with a drug delivery system, in accordance with various embodiments;

FIGS. 4a-4b illustrate an exemplary flow controller for use with a drug delivery system, in accordance with various embodiments;

FIGS. 5a-5b illustrate an exemplary flow controller for use with a drug delivery system, in accordance with various embodiments;

FIG. 6 illustrates an example take-home, disposable drug delivery system in accordance with various embodiments

FIG. 7 illustrates an exemplary flow sensor for use with a drug delivery system, in accordance with various embodiments.

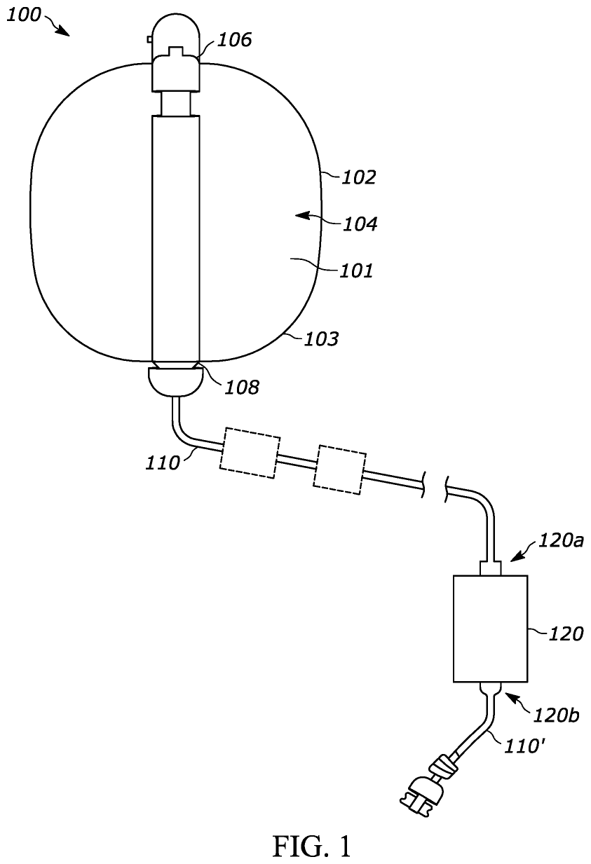

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Turning to the figures, pursuant to these various embodiments, a disposable, take-home drug delivery system 100 is provided. The drug delivery system varies from an electromechanical programmable IV pump in that the systems such as the drug delivery system 100 described herein relies primarily and/or partially on material characteristics of the pump and/or gravitational forces (as opposed to an external power source) to administer a drug to a patient. These take-home systems described herein are typically smaller, lower cost, and easier to use compared to electromechanical programmable IV pumps, and as a result, can be used in settings outside of a healthcare facility (e.g., at a patient's home, office, and/or other location). By focusing on a single therapeutic or class of therapeutics, a simpler approach to a user interface and risk assessment may be afforded, thereby potentially reducing costs of goods sold (“COGS”), power requirements, and size, thus increasing value to patients. The system 100 includes a small, energy efficient “add-on” unit that may be incorporated into a take-home pump system with minimal complexity. The system 100 may be used in intravenous, subcutaneous, intra-arterial, intramuscular, and/or epidural delivery approaches having delivery times between approximately five minutes and approximately twenty-four hours. By using the drug delivery system 100 described herein, patient anxiety and confusion is reduced due to the use of a positive pressure flow and/or gravitational flow that eliminates the need for regulatory guidance for air bubble detection as compared to peristaltic time mechanisms. The systems described herein provide an optional, single use, pre-programmed add-on unit that provides limited functionality at the patient level. Accordingly, the add-on system is simplified.

The system 100 includes a drug delivery container 102 (e.g., an intravenous drug delivery container) which could also be considered a medication reservoir that includes a container body 103 having an inner volume 104 that accommodates a drug 101 therein. The inner volume 104 may be sterile. This container 102 may be an off-the shelf disposable elastomeric pump of any desired size. In the illustrated example, the delivery container 102 also functions as the drive mechanism that causes the drug 101 to be administered to the patient.

Specifically, the container body 103 may be constructed from an elastic and/or resilient material. Generally speaking, the container body 103 is in a relaxed state prior to filling the drug 101 therein, and upon inserting the drug 101 into the container body 103, the container body 103 is expanded or stretched outwardly, and the inner volume 104 increases. The elasticity of the container body 103 generates a contraction force on the inner volume 104 that ultimately is exerted on the drug 101 for drug administration. The container 102 further includes an inlet fill port or mechanism 106 and an outlet port or mechanism 108. These ports 106, 108 may be of any type to allow for selective coupling of drug containers, vials, syringes, and the like. In some examples, the inlet fill port 106 and the outlet fill port 108 may include a valve or sealing mechanism to selectively permit fluid flow, and may be capped to prevent external contamination. Coupled to the outlet port 108 is an IV pump supply line or tubing 110 that is operably coupled to the outlet fill port 108 and dimensioned to accommodate flow of the drug 101 for patient administration. This IV supply line 110 may be an off the shelf item and may have any number of desired characteristics such as length and/or flexibility. Any number of additional components may be coupled to the IV supply line 110 such as, for example, clips, clamps, filters (e.g., air elimination filters), and the like.

Typically, healthcare professionals (e.g., clinical pharmacies) stock a variety of delivery containers 102, thereby enabling ready access to the reservoir and drive (i.e., the motive force). One such example brand of delivery containers 102 is Easy Pump (e.g., Easy Pump LT 125-5-S, LT 279-27-S, etc.) which may include inner volumes varying from approximately 15 mL to approximately 500 mL. These models may be in the form of high flow, medium flow, low flow, and/or ultra-low flow, and may result in a wide array of desired drug flow rates (e.g., between approximately 0.3 mL/day and approximately 500 mL/hour). As a result, a nominal infusion time may vary between approximately 5 minutes and approximately 60 hours depending on the desired usage.

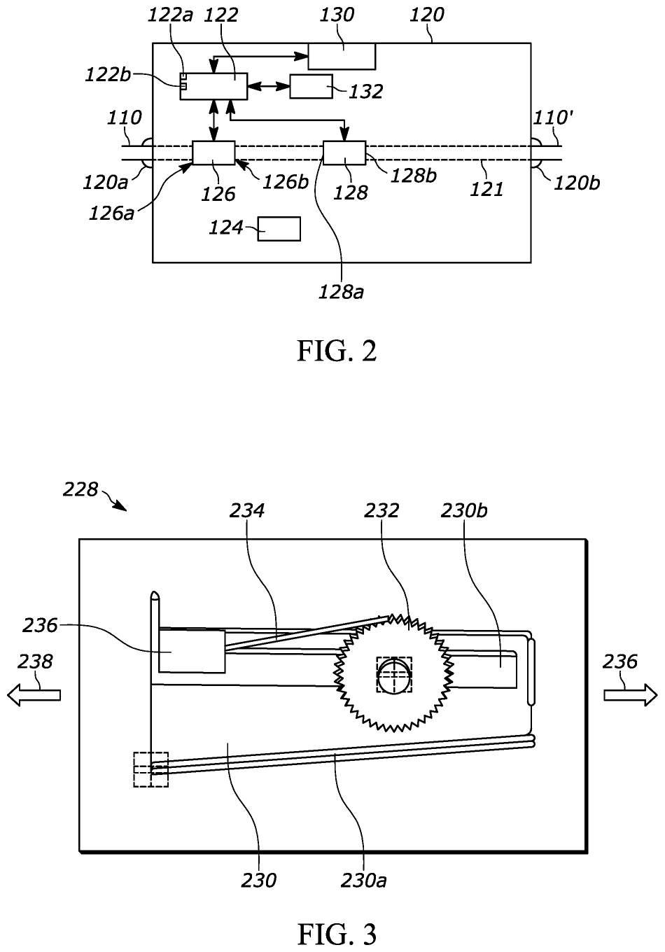

The system 100 additionally includes a flow rate monitor 120 that may be operably coupled to the IV supply line 110. In some examples, the flow rate monitor 120 may be directly coupled to the outlet port 108. The flow rate monitor 120 may include a flow rate sensor 126 that senses a flow rate of the drug 101 within the IV supply line 110. More specifically, as illustrated in FIG. 2, the flow rate monitor 120 includes a controller 122, a power source 124, the aforementioned flow rate sensor 126 operably coupled to the controller 122, and a fluid flow control device 128 operably coupled to the controller and in fluid communication with the IV supply line 110. The flow rate monitor 120 may additionally include any number of optional components such as, for example, an interface 130 and an alarm 132.

The flow rate monitor 120 may be provided with the drug delivery system 100 packaging to encourage its use (though its use is not required in the event a healthcare professional has strong preferences opposing its use). In other words, the flow rate monitor 120 may be an optional component in the take-home drug delivery system 100 that the healthcare professional and/or the patient may use as they deem appropriate. The flow rate monitor 120 may be in the form of a clamshell housing that accommodates each of the components therein, and may include an inlet 120a and an outlet 120b, and may include internal tubing 121 extending between the inlet 120a and the outlet 120b. In some examples, the IV supply line 110 is coupled to the inlet 120a, and a second supply line 110′ (that ultimately couples to a user for drug administration) is coupled to the outlet 120b. One example flow rate monitor 120 is provided by Sensirion (e.g., the Sensirion LD20-0600L single use liquid flow sensor). Another such example is the DripAssist Infusion Rate Monitor, available at www.boundtree.com. Other examples are possible.

The flow rate monitor 120 differs from complex electromechanical infusion pumps by lacking user/patient programmability. Specifically, the flow rate monitor 120 is “programmed” at a location that is upstream from the user's at-home environment (e.g., at a pharmacy prior to providing the patient with their prescription). In this sense, the flow rate monitor 120 may be viewed as a single-use, fixed programmed, pre-grammed device that only provides the patient with a limited feature set (e.g., initiate or pause dosages). Further, compared to complex electromechanical IV pumping systems, the flow rate monitor 120 described herein additionally lacks the typical programmable features afforded to healthcare professionals. In some examples, the “programmability” afforded to healthcare professionals may be limited to simply inputting the prescribed drug and/or dosage information. Accordingly, in some examples, the flow rate monitor 120 may not be reprogrammable after an initial programming.

The controller 122 includes software 122a adapted to control its operation, any number of hardware elements 122b (such as, for example, a non-transitory memory module and/or processors), any number of inputs, any number of outputs, and any number of connections. The software 122a may be loaded directly onto a non-transitory memory module of the controller 122 in the form of a non-transitory computer readable medium, or may alternatively be located remotely from the controller 122 and be in communication with the controller 122 via any number of controlling approaches. The software 122a includes logic, commands, and/or executable program instructions which may contain logic and/or commands for controlling the flow rate monitor 120. The software 122a may or may not include an operating system, an operating environment, an application environment, and/or the user interface 130.

The power source 124 may be any type of power source capable of powering the components in the flow rate monitor 120. For example, the power source 124 may be in the form of a watch or cell-battery dimensioned to power the flow rate monitor 120 during a complete administration cycle. Other examples are possible. The flow rate sensor 126 may be of any type (e.g., the aforementioned examples) and may include a sensor inlet 126a and a sensor outlet 126b that couple to the internal tubing 121 through which the drug 101 passes during administration. Upon sensing the measured flow rate, the flow rate sensor 126 transmits this data to the controller 122.

The fluid flow control device 128 may be any type of device capable of modifying the fluid flow rate of the drug 101. In some examples, the fluid flow control device 128 may be any type of valve mechanism, and in other examples, the fluid flow control device 128 may be any type of pump mechanism. The fluid flow control device 128 includes a flow device inlet 128a and outlet 128b that couple to the internal tubing 121 through which the drug 101 passes during administration. In other words, the fluid flow device 128 is disposed within the flow path of the drug 101. The fluid flow control device 128 includes at least one operational parameter that may be modified during operation of the flow rate monitor 120. In some examples, the operational parameter may be a driving or motive output (e.g., a force or power exerted by the pump or motor) or a valve position. Other examples are possible.

Upon the controller 122 receiving an input value (e.g., from the user interface 130 described below) that indicates a desired drug and/or dosage to be administered, the controller 122 initiates a risk profile corresponding to the selected drug. This risk profile may include an indication of an allowable flow rate range for the particular drug 101 being administered and/or any additional important operational values associated with the drug. The controller 122 receives the sensed flow rate from the flow rate sensor 126 and compares this value to data contained in the risk profile (e.g., a required flow rate value or range of values) to generate or calculate a difference value. If the difference value exceeds a threshold value (e.g., a range of approximately 5% from the desired flow rate or flow rate range), the controller 122 sends a signal that causes the fluid flow control device 128 to adjust the operational parameter, thus modifying the fluid flow rate of the drug 101. In some examples, it may be desirable to have the flow rate of the fluid flow control device 128 to be between an average flow rate and a full scale of the flow rate sensor 126.

The user interface 130 may include a number of inputs (e.g., buttons) and/or displays that allow a healthcare professional and/or a patient to initially configure the flow rate monitor 120. Generally, the interface 130 includes a limited number of patient-level settings and inputs to reduce user confusion. For example, a healthcare professional may use the interface 130 to input a desired flow rate, a duration of drug delivery, and/or a risk profile for the specific drug 101 being administered, and this input or inputs will be transmitted to the controller 122. In some examples, all or some of this information may be already stored on the controller 122, and thus the healthcare professional may only need to enter the drug name and/or dosage. As previously stated, the software 122a on the controller 122 may be capable of determining desired output values required to operate the flow rate monitor 120 based on the input or inputs received from the interface 130 and determine required tolerances (e.g., threshold and/or alarm values). Put another way, the interface 130 may be configured to only generate an output and may not receive any inputs beyond a selection of a desired drug.

The interface 130 may additionally include buttons that begin and/or pause operation of the system 100 so that a user may begin drug administration at a desired time. The interface 130 may also include a display that can indicates desired and/or actual flow values, error messages, remaining dosage time, and the like. In some examples, the interface may be disposed on or within the flow rate monitor 120, or optionally may be implemented via external connectivity (e.g., via a portable electronic device such as a smart phone, computer, tablet, etc.).

The optional alarm 132 may function as a feedback device to alert the user of a potential problem (e.g., a full and/or partial occlusion) in the system 100. The alarm may be in the form of a speaker that produces an audible noise, a buzzer that vibrates, and/or a light that flashes. Other examples are possible. In these examples, upon a user inputting settings (e.g., the particular drug, a desired flow rate, etc.) into the interface 130, the controller 122 may determines the appropriate risk profile, which can include an alarm value, via software 122a. In the event that the sensed flow value obtained from the sensor 126 exceeds this alarm value, the controller 122 may transmit a signal that causes the alarm 132 to be triggered and/or actuated. For example, the alarm value may be a range of approximately 10-15% from the desired flow rate. In other words, if the measured or sensed flow rate is higher or lower than 10%-15% of the desired flow rate, the alarm may be triggered, thus alerting the user to take appropriate action. Advantageously, by using the alarm 132, the patient will no long need to restart on a new delivery cycle upon occurrence of an occlusion.

In some examples, the system 100 may additionally include at least one compliance member in the form of a flexible tube, a diaphragm, and/or a bellows that predictably absorb high frequency fluid displacement operation. Some drug delivery systems operating at high frequencies (e.g., more than 50% duty cycle, or where chamber is filling for at least 50% of the time) may need a compliance member to ensure delivery accuracy. Lower frequency delivery allows sufficient time to ‘equalize’ and create predictable delivery, but for high frequencies (e.g., when using components such as a rigid flow controller system), there may be reduced accuracy after the system has completely primed and eliminated air bubbles (e.g., compliance). A compliance member positioned downstream of the flow controller will ensure delivery accuracy.

So configured, the flow rate monitor 120 may be implemented as an optional component in existing delivery systems 100 used in a variety of locations including a patient's home, office, or other non-medical facility environment. In some examples, the flow rate monitor 120 may be water resistant or waterproof to enable use while a user bathes. The flow rate monitor 120 may be provided with a coiled second supply line 110′ that automatically retracts, thus staying out of the way of the user.

Advantageously, the flow rate monitor 120 provides increased accuracy as compared to conventional reusable systems (e.g., conventional systems have an accuracy of approximately ±15%, while the system described herein may result in an accuracy of approximately ±6%) and may reduce and/or eliminate patient sensitivity to running out of drug 101. The flow rate monitor 120 may allow for a constant pressure to be delivered over longer periods of time. Further, the need to overfill the container 102 is eliminated due to less wasted medication and feedback in the case of blockage. Advantageously, alarms are minimized through the use of custom risk profile based on the specific drug 101.

The flow rate monitor 120 may be replaced at each refill interval, so battery 124 needn't occupy a large volume. Accordingly, the flow rate monitor 120 may have a small, discrete, patient-friendly size that is easy to transport and is suitable for pain management. In some examples, by pairing a relatively high flow displacement pump with the flow rate monitor 120, a low duty cycle may be provided that only allows flow for approximately 6% of the overall administration time, thereby reducing amount of time the sensor 126 needs to be powered. Most drug delivery cycles may be averaged over time such that the flow rate monitor 120 delivers numerous high flow rates for short periods of time, which is the clinical equivalent to constant, low flow rates.

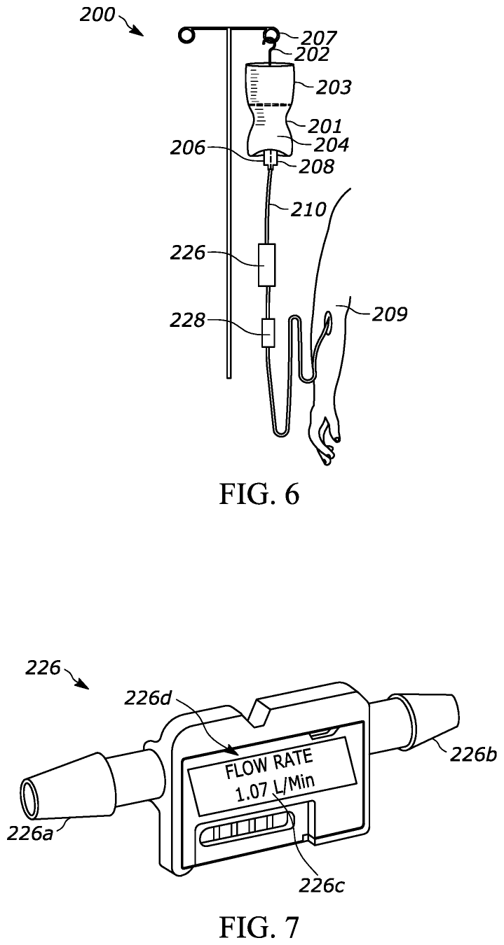

As shown in FIG. 6, the flow sensor 226 and flow controller 228 may be separate components each operatively coupled with and/or positioned along the supply line 210. FIG. 6 shows a drug delivery system 200 having a delivery container 202 having a container body 203 adapted to accommodate a drug 201 therein, an outlet port 208, an inlet 206, and a hanging portion 207 to support the container 202 such that gravitational forces urge the drug 201 from the outlet port 208. The system 200 further includes a supply line 210 operably coupled to the outlet port 208 to deliver the drug 201 to a user 209, the flow rate sensor 226 that senses a flow rate of the drug 201 within the supply line 210; and a flow controller 228 operatively coupled to at least the flow rate sensor 226. The flow controller 228 is configured to regulate a flow rate of the drug 201. The flow controller includes a supply line restrictor movable between at least a first setting and a second setting, where the supply line is more restricted when the supply line restrictor is in the second setting than in the first setting. The flow controller further includes a controller for adjusting the supply line restrictor between at least the first setting and the second setting in response to input from the flow rate sensor. In some embodiments, the controller can move the supply line restrictor continuously between the first setting and the second setting, meaning that the supply line restrictor may have a plurality of non-discreet settings located between the first and second settings, for example. The plurality of setting can be characterized, for example, as an infinite number of settings. In other versions, the supply line restrictor may alternatively have a plurality of discrete settings between the first and second settings. Furthermore, in some embodiments, the controller can move the supply line restrictor from the first setting to the second setting, as well as from the second setting to the first setting. Specifically, in some example situations, there may be more driving pressure at the beginning of an infusion and the controller would need to open up the flow path as the delivery progresses. And in the event of a partial block in the delivery line, from either clotting at the cannula or sitting/kinking the infusion line, the controller may need also to increase flow to compensate for the blockage, but at the resolution of the occlusion event, the controller would need to decrease the flow. Thus, in some embodiments, the controller can move the restrictor back and forth, perhaps multiple times in reversible directions, throughout the infusion process.

As shown in FIG. 7, an exemplary flow rate sensor 226 includes an inlet 226a and an outlet 226b that are each fluidly connected with various portions of the supply line 210 and a display 226c for displaying to the user and/or health care provider the flow rate. The flow rate sensor may also have a flow rate sensor output 226d for providing flow rate information to the flow controller 228, such as via a wireless connection. Other suitable flow rate sensors may be used, such as a sensor that does not need to be in fluid connection with the supply line 210, a flow sensor with an analog display, or other suitable flow rate sensors. For example, the system in the present disclosure may be utilized with flow rate sensors disclosed and/or described in Application No. 62/925,676, filed on Oct. 24, 2019, the contents of which are incorporated by reference.

FIG. 3 shows an exemplary flow controller 228, having a body 230, a roller clamp 232 movable with respect to the body 230, a control arm 234 configured to move the roller clamp 232, and a controller 236 configured to control the control arm 234 (and as a result to move the roller clamp 232). The flow controller 228 is operatively coupled with the flow rate sensor 226, such as via wireless communications components in the controller 236. The flow controller is configured to regulate a flow rate of the drug, such as by movement of the roller clamp 232. For example, the roller clamp 232 is a type of supply line restrictor that is movable between at least a first setting and a second setting (including continuously and non-discreetly between a plurality of settings between the first and second settings as described hereinabove), where the supply line is more restricted when the supply line restrictor is in the second setting than in the first setting. As a more specific example, the roller clamp 232 is movable along a groove 230b formed in the body 230. The groove 230b is not parallel with a bottom (ramp) surface 230a of the body 230. The supply line 210 is positioned between the roller clamp 232 and the ramp surface 230a, such that as the roller clamp 232 moves along the groove 230b, the supply line 210 will become more restricted or less restricted depending on the direction of travel. As a more specific example, as the roller clamp 232 moves in direction 236 the supply line 210 will become more restricted and have a lower flow rate of the drug. Conversely, as the roller clamp 232 moves in direction 238 the supply line 210 will become less restricted and have a higher flow rate of the drug.

The control arm 234 shown in FIG. 3 extends from the controller 236 to the roller clamp 232. For example, the control arm 234 is coupled with an outer surface of the roller clamp 232 such that transverse movement of the control arm 234 (i.e., extension or retraction of the control arm 234) moves the roller clamp 232. For example, the control arm 234 may be coupled with the roller clamp 232 by a connector pin such that the angular position of the control arm 234 with respect to the body 230 is able to change as the arm is extended or retracted from the controller 236. Additionally or alternatively, the control arm 234 may be rigid along its axis but somewhat flexible in a direction transverse to its axis. The controller 236 may include a motor (not shown) and a rotating shaft that extends and retracts the controller arm 234, such as with a geared arrangement, conveyor belt arrangement, and/or where the controller arm 234 is wrapped around the rotating shaft (to wind or unwind therefrom). The roller clamp 232 may also be manually moved by a user, as desired, to “manually override” the controller and/or to set an initial flow rate.

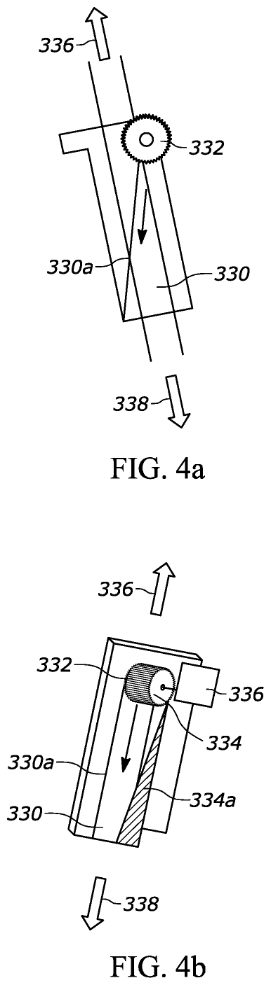

FIGS. 4a and 4b show side views of another exemplary flow controller 328, having a body 330, a roller clamp 332 movable with respect to the body 330, a geared wheel 334 coupled with the roller clamp 332, a track 334a extending along the body 330 and configured receive geared teeth of the geared wheel 334, and a controller 336 configured to control the geared wheel 334 (and as a result to move the roller clamp 332). The flow controller 328 is operatively coupled with the flow rate sensor 226, such as via wireless communications components in the controller 336. The flow controller is configured to regulate a flow rate of the drug, such as by movement of the roller clamp 332. For example, the roller clamp 332 is a type of supply line restrictor that is movable between at least a first setting and a second setting (including continuously and non-discreetly between a plurality of settings between the first and second settings as described hereinabove), where the supply line is more restricted when the supply line restrictor is in the second setting than in the first setting. As a more specific example, the roller clamp 332 is movable along a groove (not shown, similar as in FIG. 3) formed in the body 330. The groove is not parallel with a bottom (ramp) surface 330a of the body 330. The supply line 210 is positioned between the roller clamp 332 and the ramp surface 330a, such that as the roller clamp 332 moves along the groove, the supply line 210 will become more restricted or less restricted depending on the direction of travel. As a more specific example, as the roller clamp 332 moves in direction 336 the supply line 210 will become more restricted and have a lower flow rate of the drug. Conversely, as the roller clamp 332 moves in direction 338 the supply line 210 will become less restricted and have a higher flow rate of the drug.

The geared wheel 334 shown in FIGS. 4a-4b is positioned between the controller 336 and the roller clamp 332. For example, the geared wheel 334 is coupled with the roller clamp 332 such that the two components are rotationally coupled with each other (i.e., as the geared wheel 334 rotates the roller clamp 332 likewise rotates). The geared wheel 334 is also rotationally coupled with a rotor (not shown) such that rotation of the rotor likewise rotates the geared wheel 334. Alternatively, the rotor may be directly connected to the roller clamp 332 such that rotation of the rotor rotates the roller clamp 332. The geared wheel 334 has gear teeth that mate with sections of the track 334a such that as the geared wheel rotates it translates along the track 334a, in direction 336 or 338. Alternatively, the roller clamp 332 may directly mate with the track 334a (e.g., in the embodiment where the roller clamp 332 is directly coupled with the rotor). In either case, the controller 336 is preferably free to translate along the track 334a as well. Alternatively, the rotor of another portion of the controller 336 may be flexible to accommodate translational movement between the controller and the roller clamp 332. The roller clamp 332 may also be manually moved by a user, as desired, to “manually override” the controller and/or to set an initial flow rate.

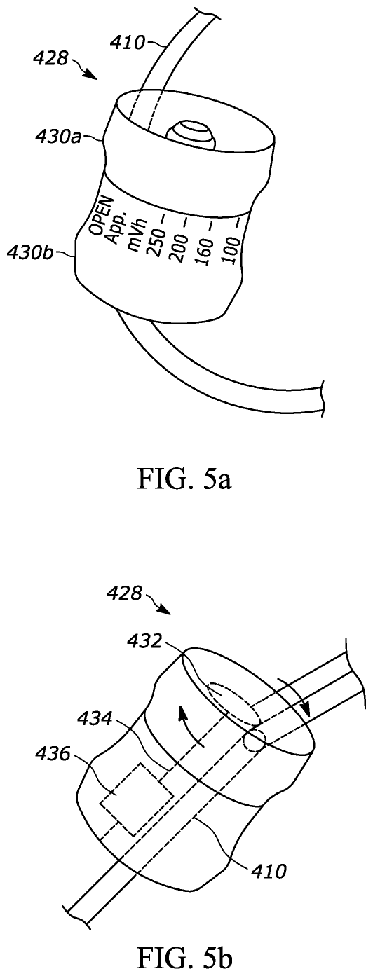

FIGS. 5a and 5b show a side and a cross-sectional view of another exemplary flow controller 428, having a body 430 having an upper portion 430a and a lower portion 430b rotatable with respect to each other, an eccentric disc 432 adjacent to the supply line 410 that is extending through the flow controller 428, and a controller 436 configured to control rotational movement of the eccentric disc 432 and/or one of the housing portions 430a, 430b. For example, in the embodiment shown in FIGS. 5a and 5b, the upper housing portion 430a, is rotatably coupled with the eccentric disc 432 such that as the upper and lower housing portions 430a, 430b rotate with respect to each other the eccentric disc 432 rotates with respect to the supply line 410 and adjusts the level of compression of the supply line 410. The flow controller 428 is operatively coupled with the flow rate sensor 226, such as via wireless communications components in the controller 436. The flow controller 428 is configured to regulate a flow rate of the drug, such as by movement of the upper portion 430a. For example, the flow controller 428 is a type of rotary dial supply line restrictor that is movable between at least a first setting and a second setting (including continuously and non-discreetly between a plurality of settings between the first and second settings as described hereinabove), where the supply line is more restricted when the supply line restrictor is in the second setting than in the first setting. As a more specific example, as the upper portion 430a rotates in one direction the supply line 410 will become more restricted and have a lower flow rate of the drug. Conversely, as the upper portion 430a rotates in the opposite direction the supply line 410 will become less restricted and have a higher flow rate of the drug.

Rotation of the eccentric disc 432 shown in FIGS. 5a-5b is controlled by the controller 436. For example, the eccentric disc 432 may be rotationally coupled with a rotor (not shown) such that rotation of the rotor likewise rotates the eccentric disc 432. Alternatively, the rotor may be directly connected to the roller clamp 332 such that rotation of the rotor rotates the roller clamp 332. Alternatively, the eccentric disc 432 may be coupled with controller arm 434 that operates similarly to the control arm discussed above with respect to the flow controller 228 in FIG. 3. The upper housing portion 430a (and thereby the eccentric disc 432) may also be manually rotated by a user, as desired, to “manually override” the controller and/or to set an initial flow rate.

Alternatively, rotation of the flow controller 428 may operate by directly constricting a flow path through the flow controller 428 rather than by compressing a supply line. For example, the upper and lower portions of the housing may define upper and lower flow paths, respectively, and such flow paths may be slightly eccentric to each other such that as the housing portions rotate with respect to each other the cross-sectional area of overlap between the upper and lower flow paths becomes smaller or larger.

As discussed above, the flow sensor 226 and the flow controller 228, 328, 428 are preferably in operable communication with each other to improve accuracy and lower tolerances for the flow rate controls. For example, one or both components may include wireless transmitters and/or receivers to facilitate wireless communication. Alternatively, or additionally, the components may be coupled via wired communication lines. In either case, the respective components are configured to deliver accurate results with low complexity and cost of goods so that the components may be disposable/one-use components.

The above description may be particularly applicable and/or beneficial when utilized with bispecific T cell engager (BITE®) antibodies such as but not limited to BLINCYTO® (blinatumomab). For example, BiTE® antibodies may need to be administered via continuous infusion. The relatively short half-life of BiTE®s may benefit from and/or necessitate a continual infusion to maintain certain plasma levels during the course of treatment. One of the challenges with this type of administration is utilizing a flow management system that presents the least issues in terms of supply complexity, such as a variable flow rate over the duration of the treatment. For example, the infusion rate may be changed depending on the stage of dosing, such as a “loading dose phase” where the infusion rate is started out slow and ramped up or an eventual “maintenance dose phase”. Utilizing variable stages may reduce a risk of cytokine release syndrome (CRS). The closed-loop flow rate control described herein may be particularly applicable and/or beneficial for BiTE® administration.

As another example, the system described herein may be particularly beneficial because it may be utilized without requiring specialty components such as the IV lines or drug containers. For example, continuous infusion pumps often require use of specialty IV lines and/or IV bags. However, the systems set forth herein may utilize many or most available IV lines and/or IV bags. This is particularly applicable and/or beneficial for BiTE® antibodies, as some BiTE®s are not compatible with all IV administration materials. For example, adsorption issues have been observed with PVC lined components.®Furthermore, some pump manufacturers do not have lines that are compatible with our drug, and this varies by region. Because current models for administration often rely on sites to use their own pump, they often have difficulty determining whether the pump has a suitable line that can be used safely with the drug. Many regional clinical sites have had trouble sourcing an appropriate line+pump combination.

However, the systems described herein may not/do not require a pump, thereby resolving many of these difficulties/problems. More specifically, infusion rates being utilized on most BiTE® programs are achievable without powered displacement of fluid. Gravity drip is used to manage many medications across the globe, and a known drug compatible IV line could be supplied instead of trying to source a pump/line combination. The “gravity drip method” for adjusting infusion rates is typically managed with a roller clamp on a standard IV line. The roller clamp is tightened/loosened to increase/decrease the flow of solution to the patient. Flow rate is currently monitored via the “drip count” method. With this method an HCP counts the number of drips over a certain time interval and uses a chart to calculate the corresponding flow rate. However, this method may not be suitably or desirably accurate, and the systems described herein may provide a more accurate flow rate control. For example, the systems described herein, which offer the accuracy of a closed loop system, may alleviate the need to manage or supply additional pumps and lines.

The above description describes various devices, assemblies, components, subsystems and methods for use related to a drug delivery device. The devices, assemblies, components, subsystems, methods or drug delivery devices can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“ID-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-?4ß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Solids™ (eculizumab); pexelizumab (anti-05 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNF? monoclonal antibody); Reopro® (abciximab, anti-GP Ilb/Ilia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-?4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2R? mAb); Zevalin® (ibritumomab tiuxetan); Zetia® (ezetimibe); Orencia® (atacicept, TALI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNF? mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-?5?1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxinl mAb (CAT-213); anti-FGF8 mAb; anti-ganglioside GD2 mAb; anti-ganglioside GM2 mAb; anti-GDF-8 human mAb (MY0-029); anti-GM-CSF Receptor mAb (CAM-3001); anti-HepC mAb (HuMax HepC); anti-IFN? mAb (MEDI-545, MDX-198); anti-IGF1R mAb; anti-IGF-1R mAb (HuMax-Inflam); anti-IL12 mAb (ABT-874); anti-IL12/1L23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-I P10 Ulcerative Colitis mAb (MDX-1100); BMS-66513; anti-Mannose Receptor/hCG? mAb (MDX-1307); anti-mesothelin dsFv-PE38 conjugate (CAT-5001); anti-PD1mAb (MDX-1106 (ONO-4538)); anti-PDGFR? antibody (IMC-3G3); anti-TGFß mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BITE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF ? monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™ or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG12C small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CART (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BITE®). In some embodiments, the drug delivery device may contain or be used with AMG 256 or another product containing an anti-PD-1×IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33×anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19×CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3×epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33×anti-CD3 BiTE® (bispecific T cell engager) construct. In some embodiments, the drug delivery device may contain or be used with AMG 701 or another product containing a half-life extended (HLE) anti-B-cell maturation antigen (BCMA)×anti-CD3 BiTE® (bispecific T cell engager) construct. In some embodiments, the drug delivery device may contain or be used with AMG 757 or another product containing a half-life extended (HLE) anti-delta-like ligand 3 (DLL3)×anti-CD3 BiTE® (bispecific T cell engager) construct. In some embodiments, the drug delivery device may contain or be used with AMG 910 or another product containing a half-life extended (HLE) epithelial cell tight junction protein claudin 18.2×CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A drug delivery system comprising:
a delivery container including a container body adapted to accommodate a drug therein, an outlet port, and a hanging portion adjacent to a top of the delivery container, the delivery container configured to be supported by the hanging portion such that gravitational forces urge the drug from the outlet port;
a supply line operably coupled to the outlet port to deliver the drug to a user;
a flow rate sensor that senses a flow rate of the drug within the supply line; and
a flow controller operatively coupled to at least the flow rate sensor, wherein the flow controller is configured to regulate a flow rate of the drug, the flow controller comprising:
a supply line restrictor movable between at least a first setting and a second setting, where the supply line is more restricted when the supply line restrictor is in the second setting than in the first setting; and
a controller for adjusting the supply line restrictor between at least the first setting and the second setting in response to input from the flow rate sensor.

2. The drug delivery system of claim 1, wherein the controller is adapted to receive the input from the flow rate sensor and compare the input to a desired flow rate to calculate a difference value, wherein upon the difference value exceeding a threshold value, the controller is adapted to adjust an operational parameter of the supply line restrictor.

3. The drug delivery system of claim 1, wherein the flow restrictor includes a ramped surface and a movable clamp, wherein a portion of the supply line is positioned between the ramped surface and the movable clamp, and wherein the flow controller includes a controller arm coupled with the movable clamp and configured to move the movable clamp between at least a first position and a second position, wherein the controller arm is optionally configured to move the movable clamp along at least a length of the ramped surface.

4. (canceled)

5. The drug delivery system of claim 1, wherein the flow restrictor further includes:
a ramped surface; and
a track extending adjacent to the ramped surface;
wherein the supply line restrictor is a movable clamp operatively engaged with the track, wherein a portion of the supply line is positioned between the ramped surface and the movable clamp, and
wherein the flow controller includes a motor configured to rotate the movable clamp and transversely move the movable clamp along the track.

6. The drug delivery system of claim 1, wherein the flow restrictor includes a rotary dial, wherein a portion of the supply line is positioned adjacent to the rotary dial, and wherein the flow controller includes an eccentric portion coupled with the rotary dial and configured to move the rotary dial between at least a first position and a second position.

7. The drug delivery system of claim 6, wherein the flow restrictor further includes a controller arm configured to rotate the rotary dial, wherein the controller arm is configured to rotate the rotary dial along at least a rotational profile of at least 30 degrees.

8-9. (canceled)

10. The drug delivery system of claim 2, wherein the flow controller further comprises an alarm operably coupled to the controller, the controller being adapted to activate the alarm upon the difference value exceeding an alarm value.

11. The drug delivery system of claim 1, wherein the flow rate sensor further comprises: 1) an interface coupled to the controller to receive at least one input; and 2) a display coupled to the controller.

12. The drug delivery device of claim 1, wherein the supply line restrictor is at least one of (a) movable continuously between a plurality of non-discreet settings between the first setting and the second setting, or (b) movable from the first setting to the second setting, and from the second setting to the first setting.

13. (canceled)

14. A drug delivery system comprising:
a delivery container including a container body adapted to accommodate a drug therein and an outlet port, the container body being constructed from a resilient material such that the container body is adapted to exert an urging force on the drug to expel the drug from the outlet port;
a supply line operably coupled to the outlet port to deliver the drug to a user;
a flow rate sensor that senses a flow rate of the drug within the supply line; and
a flow controller operatively coupled to at least the flow rate sensor, wherein the flow controller is configured to regulate a flow rate of the drug, the flow controller comprising:
a supply line restrictor movable between at least a first setting and a second setting, where the supply line is more restricted when the supply line restrictor is in the second setting than in the first setting; and
a controller for adjusting the supply line restrictor between at least the first setting and the second setting in response to input from the flow rate sensor.

15. The drug delivery system of claim 14, wherein the controller is adapted to receive the input from the flow rate sensor and compare the input to a desired flow rate to calculate a difference value, wherein upon the difference value exceeding a threshold value, the controller is adapted to adjust an operational parameter of the supply line restrictor.

16. The drug delivery system of claim 14, wherein the flow restrictor includes a ramped surface and a movable clamp, wherein a portion of the supply line is positioned between the ramped surface and the movable clamp, and wherein the flow controller includes a controller arm coupled with the movable clamp and configured to move the movable clamp between at least a first position and a second position, wherein the controller arm is optionally configured to move the movable clamp along at least a length of the ramped surface.

17. (canceled)

18. The drug delivery system of claim 14, wherein the flow restrictor further includes:
a ramped surface; and
a track extending adjacent to the ramped surface;
wherein the supply line restrictor is a movable clamp operatively engaged with the track, wherein a portion of the supply line is positioned between the ramped surface and the movable clamp, and
wherein the flow controller includes a motor configured to rotate the movable clamp and transversely move the movable clamp along the track.

19. The drug delivery system of claim 14, wherein the flow restrictor includes a rotary dial, wherein a portion of the supply line is positioned adjacent to the rotary dial, and wherein the flow controller includes an eccentric portion coupled with the rotary dial and configured to move the rotary dial between at least a first position and a second position.

20. The drug delivery system of claim 19, wherein the flow restrictor further includes a controller arm configured to rotate the rotary dial, wherein the controller arm is optionally configured to rotate the rotary dial along at least a rotational profile of 30 degrees or 45 degrees.

21-22. (canceled)

23. The drug delivery system of claim 15, wherein the flow controller further comprises an alarm operably coupled to the controller, the controller being adapted to activate the alarm upon the difference value exceeding an alarm value.

24. The drug delivery system of claim 23, wherein the alarm value is at least partially based on a risk profile of the drug contained in the delivery container.

25. The drug delivery system of claim 14, wherein the flow rate sensor further comprises: 1) an interface coupled to the controller to receive at least one input; and 2) a display coupled to the controller.

26. The drug delivery system of claim 14, wherein the delivery container comprises a balloon.

27. The drug delivery device of claim 14, wherein the supply line restrictor is at least one of (a) movable continuously between a plurality of non-discreet settings between the first setting and the second setting, or (b) movable from the first setting to the second setting, and from the second setting to the first setting.

28. (canceled)
